(19) United States
(12) Patent Application Publication
(10) Pub. No.: US 2019/0343734 A1
(43) Pub. Date: Nov. 14, 2019

(54) COMPOSITIONS CONTAINING LATEX PARTICLES AND UV ABSORBERS

(71) Applicants: Rohm and Haas Company, Collegeville, PA (US); Union Carbide Chemicals & Plastics Technology LLC, Midland, MI (US)

(72) Inventors: Inna Shulman, Langhorne, PA (US); Wenjun Xu, Pottstown, PA (US); Fanwen Zeng, Audubon, PA (US)

(21) Appl. No.: 16/466,854

(22) PCT Filed: Jan. 25, 2018
(86) PCT No.: PCT/US2018/015138

§ 371 Date: Jun. 5, 2019

Related U.S. Application Data
(60) Provisional application No. 62/452,678, filed on Jan. 31, 2017.

(51) Int. Cl. A61K 8/02; A61K 8/81; A61Q 17/04

(52) U.S. Cl. CPC A61K 8/0279; A61K 8/8152; A61K 8/8111; A61K 8/8158; A61K 8/8194; A61K 2800/654; A61K 2800/412; A61K 2800/594; A61K 2800/624; A61K 2800/63; A61Q 17/04

(57) ABSTRACT

Provided are personal care compositions comprising a core polymer, at least one inner shell polymer, and an outer shell polymer providing SPF boosting and opacity, wherein the core polymer comprises polymerized units derived from monoethylenically unsaturated monomers containing at least one carboxylic acid group and non-ionic ethylenically unsaturated monomers, and the inner and outer shell polymers comprise polymerized units derived from non-ionic ethylenically unsaturated monomers and aliphatic monomers selected from the group consisting of allyl acrylate, allyl methacrylate, and mixtures thereof.

### FIELD OF THE INVENTION

This invention relates generally to personal care compositions comprising voided latex particles and UV radiation-absorbing agents.

### BACKGROUND

Personal care compositions contain a variety of additives that provide a wide array of benefits to users. Sunscreen compositions in particular contain additives that offer protection from ultraviolet (“UV”) radiation, which can damage the skin. UV radiation can be classified as UVA (long wave; i.e., wavelengths of 320-400 nm) and UVB (short wave; i.e., wavelengths of 290 to 320 nm). The efficacy of sunscreen formulations is measured by its sun protection factor (“SPF”). Since both UVA and UVB forms of radiation are harmful, sunscreen formulations offer protection from both kinds of rays. UV absorbing agents include physical blockers, such as titanium dioxide, and chemical absorbers, such as para-aminobenzoic acid and octyl methoxycinnamate. In certain compositions, it is desirable to decrease the level of UV absorbing agents due to undesirable aesthetic and toxicological effects.

To that end, personal care compositions comprising light scatterers and UV radiation-absorbing agents have been disclosed. For example, U.S. Pat. No. 5,663,213 discloses a method of improving UV radiation absorption of a composition containing at least one UV radiation absorbing agent by incorporating a voided latex particle into the composition. Although the prior art discloses such particles for use in boosting the SPF of a composition in combination with a UV absorbing agent, there is a need in the art for improved compositions having reduced amounts of styrene in the outer shell of such particles, while maintaining SPF performance and opacity.

Consequently, there is a need to develop new personal care compositions including UV absorbing agents and particle light scatterers, i.e., voided latex particles, that have an SPF boosting effect and opacity, while also minimizing the amount of styrene in the outer shell of such particles.

### STATEMENT OF INVENTION

One aspect of the invention provides a personal care composition comprising (A) voided latex particles comprising (i) a core polymer comprising polymerized units derived from (a) 20 to 60 weight % of monoethylenically unsaturated monomers containing at least one carboxylic acid group, based on the total weight of the core polymer, and (b) 40 to 80 weight % of non-ionic ethylenically unsaturated monomers, based on the total weight of the core polymer, (ii) at least one inner shell polymer comprising polymerized units derived from (a) 90 to 99.5 weight % of non-ionic ethylenically unsaturated monomers, based on the total weight of the inner shell polymer(s), and (b) 0.5 to 10 weight % of aliphatic monomers selected from the group consisting of allyl acrylate, allyl methacrylate, and mixtures thereof, based on the total weight of the inner shell polymer(s), and (iii) an outer shell polymer comprising polymerized units derived from (a) 20 to 45 weight % of non-ionic ethylenically unsaturated monomers, based on the total weight of the outer shell polymer, and (b) 55 to 90 weight % of aliphatic monomers selected from the group consisting of allyl acrylate, allyl methacrylate, and mixtures thereof, based on the total weight of the outer shell polymer, and (B) at least one UV absorbing agent, wherein the voided latex particles are present in an amount of from 0.5 to 20 weight %, based on the total weight of the composition, and wherein the voided latex particles contain a void and have a particle size of from 400 nm to 900 nm, and wherein the voided latex particles contain less than 10 weight % of styrene, based on the total weight of the particle.

Another aspect of the invention provides a method for protecting skin from UV damage, comprising topically administering to the skin an effective amount of a personal care composition comprising (A) voided latex particles comprising (i) a core polymer comprising polymerized units derived from (a) 20 to 60 weight % of monoethylenically unsaturated monomers containing at least one carboxylic acid group, based on the total weight of the core polymer, and (b) 40 to 80 weight % of non-ionic ethylenically unsaturated monomers, based on the total weight of the core polymer, (ii) at least one inner shell polymer comprising polymerized units derived from (a) 90 to 99.5 weight % of non-ionic ethylenically unsaturated monomers, based on the total weight of the inner shell polymer(s), and (b) 0.5 to 10 weight % of aliphatic monomers selected from the group consisting of allyl acrylate, allyl methacrylate, and mixtures thereof, based on the total weight of the inner shell polymer(s), and (iii) an outer shell polymer comprising polymerized units derived from (a) 20 to 45 weight % of non-ionic ethylenically unsaturated monomers, based on the total weight of the outer shell polymer, and (b) 55 to 90 weight % of aliphatic monomers selected from the group consisting of allyl acrylate, allyl methacrylate, and mixtures thereof, based on the total weight of the outer shell polymer, and (B) at least one UV absorbing agent, wherein the voided latex particles are present in an amount of from 0.5 to 20 weight %, based on the total weight of the composition, and wherein the voided latex particles contain a void and have a particle size of from 400 nm to 900 nm, and wherein the voided latex particles contain less than 10 weight % of styrene, based on the total weight of the particle.

In another aspect, the invention provides a method for boosting the SPF or UV absorption of a sunscreen composition comprising adding to said composition from 0.5 to 20 weight % of voided latex particles, based on the total weight of the composition, wherein the voided latex particles comprise (A) voided latex particles comprising (i) a core polymer comprising polymerized units derived from (a) 20 to 60 weight % of monoethylenically unsaturated monomers containing at least one carboxylic acid group, based on the total weight of the core polymer, and (b) 40 to 80 weight % of non-ionic ethylenically unsaturated monomers, based on the total weight of the core polymer, (ii) at least one inner shell polymer comprising (a) 90 to 99.5 weight % of non-ionic ethylenically unsaturated monomers, based on the total weight of the inner shell polymer(s), and (b) 0.5 to 10 weight % of aliphatic monomers selected from the group consisting of allyl acrylate, allyl methacrylate, and mixtures thereof, based on the total weight of the inner shell polymer(s), and (iii) an outer shell polymer comprising polymerized units derived from (a) 20 to 45 weight % of non-ionic ethylenically unsaturated monomers, based on the total weight of the outer shell polymer, and (b) 55 to 90 weight % of aliphatic monomers selected from the group consisting of allyl acrylate, allyl methacrylate, and mixtures thereof, based on the total weight of the outer shell polymer, and (B) at least one UV absorbing agent, wherein the voided latex particles are present in an amount of from 0.5 to 20 weight %, based on the total weight of the composition, and wherein the voided latex particles contain a void and have a particle size of from 400 nm to 900 nm, and wherein the voided latex particles contain less than 10 weight % of styrene, based on the total weight of the particle.

### DETAILED DESCRIPTION

The inventors have now surprisingly found that voided latex particles comprising a core polymer, at least one inner shell polymer, and an outer shell polymer provide SPF boosting and opacity, wherein the core polymer comprises polymerized units derived from monoethylenically unsaturated monomers containing at least one carboxylic acid group and non-ionic ethylenically unsaturated monomers, and the inner shell and outer shell polymers comprises polymerized units derived from non-ionic ethylenically unsaturated monomers and aliphatic monomers selected from the group consisting of allyl acrylate, allyl methacrylate, and mixtures thereof, wherein the voided latex particles contain less than 10 weight % of styrene, based on the total weight of the particle.

In the present invention, “personal care” is intended to refer to cosmetic and skin care compositions for leave on application to the skin including, for example, lotions, creams, gels, gel creams, serums, toners, wipes, masks, liquid foundations, make-ups, tinted moisturizer, oils, face/body sprays, topical medicines, and sunscreen compositions. “Sunscreen compositions” refers to compositions that protect the skin from UV damage. “Personal care” relates to compositions to be topically administered (i.e., not ingested). Preferably, the personal care composition is cosmetically acceptable. “Cosmetically acceptable” refers to ingredients typically used in personal care compositions, and is intended to underscore that materials that are toxic when present in the amounts typically found in personal care compositions are not contemplated as part of the present invention. The compositions of the invention may be manufactured by processes well known in the art, for example, by means of conventional mixing, dissolving, granulating, emulsifying, encapsulating, entrapping or lyophilizing processes.

As used herein, the term “polymer” refers to a polymeric compound prepared by polymerizing monomers, whether of the same or a different type. The generic term “polymer” includes the terms “homopolymer,” “copolymer,” and “terpolymer.” As used herein, the term “polymerized units derived from” refers to polymer molecules that are synthesized according to polymerization techniques wherein a product polymer contains “polymerized units derived from” the constituent monomers which are the starting materials for the polymerization reactions. As used herein, the term “(meth)acrylic” refers to either acrylic or methacrylic. As used herein, the term “(meth)acrylate” refers to either acrylate or methacrylate.

As used herein, the terms “glass transition temperature” or “T_{g}” refers to the temperature at or above which a glassy polymer will undergo segmental motion of the polymer chain. Glass transition temperatures of a polymer can be estimated by the Fox equation (Bulletin of the American Physical Society, 1 (3) Page 123 (1956)) as follows:

1/T_{g}=w₁/T_{g(1)}+w₂/T_{g(2)}

For a copolymer, w₁ and w₂ refer to the weight fraction of the two comonomers, and T_{g(1)} and T_{g(2)} refer to the glass transition temperatures of the two corresponding homopolymers made from the monomers. For polymers containing three or more monomers, additional terms are added (wₙ/T_{g(n)}). The T_{(g)} of a polymer can also be calculated by using appropriate values for the glass transition temperatures of homopolymers, which may be found, for example, in “Polymer Handbook,” edited by J. Brandrup and E. H. Immergut, Interscience Publishers. The T_{g} of a polymer can also be measured by various techniques, including, for example, differential scanning calorimetry (“DSC”). The values of T_{g} reported herein are measured by DSC.

The inventive personal care compositions contain voided latex particles. Voided latex particles useful in the invention comprise a multistaged particle containing a core polymer, at least one inner shell polymer, and an outer shell polymer. The ratio of the core weight to the total polymer weight is from 1:4 (25% core) to 1:100 (1% core), and preferably from 1:8 (12% core) to 1:50 (2% core).

The core polymer includes polymerized units derived from monoethylenically unsaturated monomers containing at least one carboxylic acid group, and non-ionic ethylenically unsaturated monomers. The core polymer may be obtained, for example, by the emulsion homopolymerization of the monoethylenically unsaturated monomer containing at least one carboxylic acid group or by copolymerization of two or more of the monoethylenically unsaturated monomers containing at least one carboxylic acid group. In certain embodiments, the monoethylenically unsaturated monomer containing at least one carboxylic acid group is copolymerized with one or more non-ionic (that is, having no ionizable group) ethylenically unsaturated monomers. While not wishing to be bound by theory, it is believed that the presence of the ionizable acid group makes the core swellable by the action of a swelling agent, such as an aqueous or gaseous medium containing a base to partially neutralize the acid core polymer and cause swelling by hydration.

Suitable monoethylenically unsaturated monomers containing at least one carboxylic acid group of the core polymer include, for example, (meth)acrylic acid, (meth)acryloxypropionic acid, itaconic acid, aconitic acid, maleic acid, maleic anhydride, fumaric acid, cronotic acid, citraconic acid, maleic anhydride, monomethyl maleate, monomethyl fumarate, monomethyl itaconate, and other derivatives such as corresponding anhydride, amides, and esters. In certain preferred embodiments, the monoethylenically unsaturated monomers containing at least one carboxylic acid group are selected from acrylic acid and methacrylic acid. In certain embodiments, the core comprises polymerized units of monoethylenically unsaturated monomers containing at least one carboxylic acid group in an amount of from 20 to 60 weight %, preferably from 30 to 50 weight %, and more preferably from 35 to 45 weight %, based on the total weight of the core polymer.

Suitable non-ionic ethylenically unsaturated monomers of the core polymer include, for example, ethylene, vinyl acetate, vinyl chloride, vinylidene chloride acrylonitrile, (meth)acrylamide, methyl (meth)acrylate, ethyl (meth)acrylate, n-butyl (meth)acrylate, i-butyl (meth)acrylate, t-butyl (meth)acrylate, cyclo-hexyl (meth)acrylate, 2-ethylhexyl (meth)acrylate, lauryl (meth)acrylate, oleyl (meth)acrylate, palmityl (meth)acrylate, and stearyl (meth)acrylate. In certain preferred embodiments, the non-ionic ethylenically unsaturated monomers are selected from methyl methacrylate and butyl methacrylate. In certain embodiments, the core comprises polymerized units of non-ionic ethylenically unsaturated monomers in an amount of from 40 to 80 weight %, preferably from 50 to 70 weight %, and more preferably from 55 to 65 weight %, based on the total weight of the core polymer.

The voided latex particles suitable for use in the present invention also include at least one inner shell polymer and an outer shell polymer, collectively referred to as “shell polymers.” The shell polymers comprise polymerized units derived from non-ionic ethylenically unsaturated monomers and aliphatic monomers selected from the group consisting of allyl acrylate, allyl methacrylate, and mixtures thereof. In certain embodiments, the shell portion of the voided latex particles are polymerized in at least two stages, and more preferably in at least three stages. As used herein, the term “outer shell polymer” refers to the composition of the final distinct polymerization stage used to prepare the voided latex particles. In certain embodiments wherein the outer shell polymer is provided by a multistage polymerization process, the outer shell polymer comprises at least 25 weight %, preferably at least 35 weight %, and more preferably at least 45 weight % of the total shell portion of the voided latex particle.

In certain embodiments, the at least one inner shell polymer(s) comprises polymerized units of aliphatic monomers selected from the group consisting of allyl acrylate, allyl methacrylate, and mixtures thereof in an amount of from 0.5 to 10 weight %, preferably from 1 to 8 weight %, and more preferably from 2 to 6 weight %, based on the total weight of the inner shell polymer(s). In certain embodiments, the at outer shell polymer comprises polymerized units of aliphatic monomers selected from the group consisting of allyl acrylate, allyl methacrylate, and mixtures thereof in an amount of from 55 to 90 weight %, preferably from 57.5 to 80 weight %, and more preferably from 60 to 75 weight %, based on the total weight of the outer shell polymer.

Suitable non-ionic ethylenically unsaturated monomers for the shell polymers include, for example, vinyl acetate, acrylonitrile, methacrylonitrile, nitrogen containing ring compound unsaturated monomers, ethylenic monomers and selected (meth)acrylic acid derivatives. Suitable (meth)acrylic acid derivatives include, for example, (C₁-C₂₂)alkyl (meth)acrylate, substituted (meth)acrylate, and substituted (meth)acrylamide monomers. In certain preferred embodiments, the (meth)acrylic acid derivatives are selected from methyl acrylate, methyl methacrylate, ethyl acrylate, ethyl methacrylate, butyl acrylate, butyl methacrylate, isobutyl acrylate, isobutyl methacrylate, hydroxyethyl methacrylate, hydroxypropyl methacrylate, dimethylaminoethyl methacrylate, dimethylaminopropyl methacrylamide, and mixtures thereof. In certain preferred embodiments, the non-ionic ethylenically unsaturated monomers comprise at least one of acrylonitrile, (meth)acrylamide, methyl (meth)acrylate, ethyl (meth)acrylate, n-butyl (meth)acrylate, i-butyl (meth)acrylate, t-butyl (meth)acrylate, cyclo-hexyl (meth)acrylate, 2-ethylhexyl (meth)acrylate, lauryl (meth)acrylate, oleyl (meth)acrylate, palmityl (meth)acrylate, stearyl (meth)acrylate, and iso-bornyl (meth)acrylate. In certain preferred embodiments, the non-ionic ethylenically unsaturated monomers comprise methyl (meth)acrylate.

In certain embodiments, the at least one inner shell polymer comprises polymerized units of non-ionic ethylenically unsaturated monomers in an amount of from 90 to 99.5 weight %, preferably from 92 to 98 weight %, and more preferably from 94 to 96 weight %, based on the total weight of the inner shell polymer(s). In certain embodiments, the outer shell polymer comprises polymerized units of non-ionic ethylenically unsaturated monomers in an amount of from 20 to 45 weight %, preferably from 22.5 to 42.5 weight %, and more preferably from 25 to 40 weight %, based on the total weight of the outer shell polymer.

In certain embodiments, the shell polymers optionally comprise polymerized units derived from at least one of monoethylenically unsaturated monomers containing at least one carboxylic acid group. Suitable monoethylenically unsaturated monomers containing at least one carboxylic acid group for the shell polymer(s) include, for example, (meth)acrylic acid, (meth)acryloxypropionic acid, itaconic acid, aconitic acid, maleic acid, fumaric acid, crotonic acid, citraconic acid, maleic anhydride monomethyl maleate, monomethyl fumarate, and monomethyl itaconate, and other derivatives such as corresponding anhydride, amides, and esters. In certain preferred embodiments, the monoethylenically unsaturated monomers containing at least one carboxylic acid group are selected from acrylic acid and methacrylic acid. In certain embodiments, the shell polymers comprise polymerized units of monoethylenically unsaturated monomers containing at least one carboxylic acid group in an amount of from 0.1 to 10 weight %, preferably from 0.3 to 7.5 weight %, and more preferably from 0.5 to 5 weight %, based on the total weight of the shell polymers.

In certain embodiments, the outer shell polymer optionally comprise polymerized units derived from at least one of monoethylenically unsaturated monomers containing at least one “non-carboxylic” acid group. Suitable monoethylenically unsaturated monomers containing at least one “non-carboxylic” acid group for the outer shell polymer include, for example, allylsulfonic acid, allylphosphonic acid, allyloxybenzenesulfonic acid, 2-acrylamido-2-methylpropanesulfonic acid (the acryonym “AMPS” for this monomer is a trademark of Lubrizol Corporation, Wickliffe, Ohio, USA), 2-hydroxy-3-(2-propenyloxy)propanesulfonic acid, 2-methyl-2-propene-1-sulfonic acid, 2-methacrylamido-2-methyl-1-propanesulfonic acid, 3-methacrylamido-2-hydroxy-1-propanesulfonic acid, 3-sulfopropyl acrylate, 3-sulfopropyl methacrylate, isopropenylphosphonic acid, vinylphosphonic acid, phosphoethyl methacrylate, styrenesulfonic acid, vinylsulfonic, acid and the alkali metal and ammonium salts thereof. In certain preferred embodiments, the monoethylenically unsaturated monomers containing at least one “non-carboxylic” acid group are selected from 2-acrylamido-2-methylpropanesulfonic acid, styrenesulfonic acid, and sodium styrene sulfonate. In certain embodiments, the outer shell polymer comprises polymerized units of monoethylenically unsaturated monomers containing at least one “non-carboxylic” acid group in an amount of from 0.1 to 10 weight %, preferably from 0.5 to 7.5 weight %, and more preferably from 1 to 5 weight %, based on the total weight of the outer shell polymer.

In certain embodiments, the voided latex particles suitable for use in the present invention contain less than 10 weight % of styrene, preferably less than 5 weight % of styrene, and more preferably less than 2.5 weight % of styrene. In certain embodiments, the voided latex particles suitable for use in the present invention are substantially free of styrene. As used herein, “substantially free of styrene” means less than 0.001 weight %, preferably less than 0.0001 weight %, and more preferably less than 1 ppm of styrene.

The shell polymers of the latex particles suitable for use in the present invention have T_{g} values which are high enough to support the void within the latex particle. In certain embodiments, the T_{g} values of at least one shell are greater than 50° C., preferably greater than 60° C., and more preferably greater than 70° C.

In certain embodiments, the core polymer and shell polymers are made in a single polymerization step. In certain other embodiments, the core polymer and shell polymers are made in a sequence of polymerization steps. Suitable polymerization techniques for preparing the voided latex particles contained in the inventive personal care compositions include, for example, sequential emulsion polymerization. In certain embodiments, the monomers used in the emulsion polymerization of the shell polymer of the voided latex particles comprise one or more non-ionic ethylenically unsaturated monomer. Aqueous emulsion polymerization processes typically are conducted in an aqueous reaction mixture, which contains at least one monomer and various synthesis adjuvants, such as the free radical sources, buffers, and reductants in an aqueous reaction medium. In certain embodiments, a chain transfer agent may be used to limit molecular weight. The aqueous reaction medium is the continuous fluid phase of the aqueous reaction mixture and contains more than 50 weight % water and optionally one or more water miscible solvents, based on the weight of the aqueous reaction medium. Suitable water miscible solvents include, for example, methanol, ethanol, propanol, acetone, ethylene glycol ethyl ethers, propylene glycol propyl ethers, and diacetone alcohol.

In certain embodiments, the void of the latex particles is prepared by swelling the core with a swelling agent containing one or more volatile components. The swelling agent permeates the shell to swell the core. The volatile components of the swelling agent can then be removed by drying the latex particles, causing a void to be formed within the latex particles. In certain embodiments, the swelling agent is an aqueous base. Suitable aqueous bases useful for swelling the core include, for example, ammonia, ammonium hydroxide, alkali metal hydroxides, such as sodium hydroxide, or a volatile amine such as trimethylamine or triethylamine. In certain embodiments, the voided latex particles are added to the composition with the swelling agent present in the core. When the latex particles are added to the composition with the swelling agent present in the core, the volatile components of the swelling agent will be removed upon drying of the composition. In certain other embodiments, the voided latex particles are added to the composition after removing the volatile components of the swelling agent.

In certain embodiments, the voided latex particles contain a void with a void fraction of from 1% to 70%, preferably from 5% to 50%, more preferably from 10% to 40%, and even more preferably from 25% to 35%. The void fractions are determined by comparing the volume occupied by the latex particles after they have been compacted from a dilute dispersion in a centrifuge to the volume of non-voided particles of the same composition. In certain embodiments, the voided latex particles have a particle size of from 400 nm to 900 nm, preferably from 450 nm to 800 nm, more preferably from 500 nm to 700 nm, and even more preferably from 550 nm to 650 nm, as measured by a Brookhaven BI-90.

A person of ordinary skill in the art can readily determine the effective amount of the voided latex particles that should be used in a particular composition in order to provide the benefits described herein (e.g., maintained UV absorption while providing a more pleasing odor profile when applied to skin), via a combination of general knowledge of the applicable field as well as routine experimentation where needed. By way of non-limiting example, the amount of voided latex particles in the composition of the invention may be in the range of from 0.5 to 20 solids weight %, preferably from 1 to 10 solids weight %, more preferably from 1 to 5 solids weight %, based on the total weight of the composition.

The personal care compositions of the present invention also comprise at least one UV absorbing agent. Suitable UV absorbing agents include, for example, oxybenzone, dioxybenzone, sulisobenzone, menthyl anthranilate, para-aminobenzoic acid, amyl paradimethylaminobenzoic acid, octyl para-dimethylaminobenzoate, ethyl 4-bis (hydroxypropyl) para-aminobenzoate, polyethylene glycol (PEG-25) para-aminobenzoate, ethyl 4-bis (hydroxypropyl) aminobenzoate, diethanolamine para-methyoxycinnamate, 2-ethoxyethyl para-methoxycinnamate, ethylhexyl para-methoxycinnamate, octyl para-methoxycinnamate, isoamyl para-methoxycinnamate, 2-ethylhexyl-2-cyano-3,3-diphenyl-acrylate, 2-ethylhexyl salicylate, homomenthyl salicylate, glyceryl aminobenzoate, triethanolamine salicylate, digalloyl trioleate, lawsone with dihydroxyacetone, 2-phenylbenzimidazole-5-sulfonic acid, 4-methylbenzylidine camphor, avobenzone, titanium dioxide, and zinc oxide. Alternatively, UV absorbing agents such as triazines, benzotriazoles, vinyl group-containing amides, cinnamic acid amides and sulfonated benzimidazoles may also be used. In certain embodiments, the personal care compositions include UV absorbing agents in an amount of from 0.1 to 50 weight %, preferably 5 to 40 weight %, and more preferably 10 to 30 weight %, based on the total weight of the composition.

Compositions of the invention also include a dermatologically acceptable carrier. Such material is typically characterized as a carrier or a diluent that does not cause significant irritation to the skin and does not negate the activity and properties of active agent(s) in the composition. Examples of dermatologically acceptable carriers that are useful in the invention include, without limitation, water, such as deionized or distilled water, emulsions, such as oil-in-water or water-in-oil emulsions, alcohols, such as ethanol, isopropanol or the like, glycols, such as propylene glycol, glycerin or the like, creams, aqueous solutions, oils, ointments, pastes, gels, lotions, milks, foams, suspensions, powders, or mixtures thereof. In some embodiments, the composition contains from about 99.99 to about 50 percent by weight of the dermatologically acceptable carrier, based on the total weight of the composition.

The personal care composition of the invention may also include, for instance, a thickener, additional emollients, an emulsifier, a humectant, a surfactant, a suspending agent, a film forming agent, a lower monoalcoholic polyol, a high boiling point solvent, a propellant, a mineral oil, silicon feel modifiers, or mixtures thereof. The amount of optional ingredients effective for achieving the desired property provided by such ingredients can be readily determined by one skilled in the art.

Other additives may be included in the compositions of the invention such as, but not limited to, abrasives, absorbents, aesthetic components such as fragrances, pigments, colorings/colorants, essential oils, skin sensates, astringents (e.g., clove oil, menthol, camphor, eucalyptus oil, eugenol, menthyl lactate, witch hazel distillate), preservatives, anti-caking agents, a foam building agent, antifoaming agents, antimicrobial agents (e.g., iodopropynyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, and vitamins (e.g., Vitamin C) and derivatives thereof. The amount of option ingredients effective for achieving the desired property provided by such ingredients can be readily determined by one skilled in the art.

As noted above, skin care compositions of the present invention are highly effective as SPF and UV absorption boosters. Accordingly, the skin care compositions of the present invention are useful for the treatment and protection of skin, including, for example, protection from UV damage, moisturization of the skin, prevention and treatment of dry skin, protection of sensitive skin, improvement of skin tone and texture, masking imperfections, and inhibition of trans-epidermal water loss. Thus, in one aspect the present invention provides that the personal care compositions may be used in a method for protecting skin from UV damage comprising topically administering to the skin a composition comprising (a) 0.1 to 50 weight % inorganic metal oxide particles, based on the weight of the composition, and (b) 0.5 to 50 weight % of a UV absorbing agent, based on the weight of the composition. The compositions may also be used in a method for boosting the SPF or UV absorption of a sunscreen composition containing a UV absorbing agent and the voided latex particles as described herein.

In practicing the methods of the invention, the skin care compositions are generally administered topically by applying or spreading the compositions onto the skin. A person of ordinary skill in the art can readily determine the frequency with which the compositions should be applied. The frequency may depend, for example, on the level of exposure to UV light that an individual is likely to encounter in a given day and/or the sensitivity of the individual to UV light. By way of non-limiting example, administration on a frequency of at least once per day may be desirable.

Some embodiments of the invention will now be described in detail in the following Examples.

### EXAMPLES

### Example 1

### Preparation of Exemplary and Comparative Copolymer Particles

Exemplary voided latex particles according to the present invention and comparative particles contain a core polymer, first inner shell polymer, second inner shell polymer, and third outer shell polymer, in the amount of 4.7 weight %, 22.1 weight %, 26.8 weight %, and 46.4 weight %, respectively, by total weight of the particles. The exemplary and comparative particles all contain the same monomer composition of the core and first inner shell, as recited in Table 1.

TABLE 1
Core and First Inner Shell of Exemplary and Comparative Particles
Monomer (wt %)

| | |
|---|---|
| Core (4.7%): | 60 MMA/40 MAA |
| 1^{st} Inner Shell (22.1%): | 8.5 BMA/88.5 MMA/3 MAA |

MMA = methyl methacrylate
MAA = methacrylic acid
BMA = butyl methacrylate

The composition of the second inner shell and outer shell of the exemplary and comparative particles contain the monomer compositions recited in Table 2.

TABLE 2
Second Inner Shell and Outer Shell of
Exemplary and Comparative Particles

| Sample | Monomer (wt %) |
|---|---|
| P-E1 | 2^{nd} Inner Shell (26.6 wt %): 94.9 MMA/5.1 ALMA |
| | Outer Shell (46.4 wt %): 22.3 MMA/75 ALMA/2.7 SSS |
| P-E2 | 2^{nd} Inner Shell (26.6 wt %): 99.25 MMA/0.75 ALMA |
| | Outer Shell (46.4 wt %): 22.3 MMA/75 ALMA/2.7 SSS |
| P-E3 | 2^{nd} Inner Shell (26.6 wt %): 98.5 MMA/1.5 ALMA |
| | Outer Shell (46.4 wt %): 22.3 MMA/75 ALMA/2.7 SSS |
| P-E4 | 2^{nd} Inner Shell (26.6 wt %): 99.25 MMA/0.75 ALMA |
| | Outer Shell (46.4 wt %): 22.3 Sty/75 ALMA/2.7 SSS |
| P-E5 | 2^{nd} Inner Shell (26.6 wt %): 98.5 MMA/1.5 ALMA |
| | Outer Shell (46.4 wt %): 37.3 Sty/60 ALMA/2.7 SSS |
| P-E6 | 2^{nd} Inner Shell (26.6 wt %): 99.25 MMA/0.75 ALMA |
| | Outer Shell (46.4 wt %): 22.3 MMA/75 AMLA/2.7 SSS |
| P-E7 | 2^{nd} Inner Shell (26.6 wt %): 98.5 MMA/1.5 ALMA |
| | Outer Shell (46.4 wt %): 22.3 MMA/75 ALMA/2.7 SSS |
| P-E8 | 2^{nd} Inner Shell (26.6 wt %): 96.9 MMA/3.1 ALMA |
| | Outer Shell (46.4 wt %): 37.3 MMA/60 ALMA/2.7 SSS |
| P-E9 | 2^{nd} Inner Shell (26.6 wt %): 99.25 MMA/0.75 ALMA |
| | Outer Shell (46.4 wt %): 37.3 MMA/60.0 ALMA/2.7 SSS |
| P-E10 | 2^{nd} Inner Shell (26.6 wt %): 96.9 MMA/3.1 ALMA |
| | Outer Shell (46.4 wt %): 37.3 MMA/60.0 ALMA/2.7 SSS |
| P-E11 | 2^{nd} Inner Shell (26.6 wt %): 99.25 MMA/0.75 ALMA |
| | Outer Shell (46.4 wt %): 37.3 MMA/60.0 ALMA/2.7 SSS |
| P-C1* | 2^{nd} Inner Shell (26.6 wt %): 94.9 Sty/5.1 DVB |
| | Outer Shell (46.4 wt %): 46.2 Sty/51.1 DVB/2.7 SSS |
| P-C2* | 2^{nd} Inner Shell (26.6 wt %): 96.9 MMA/3.1 ALMA |
| | Outer Shell (46.4 wt %): 47.3 MMA/50.0 ALMA/2.7 SSS |
| P-C3* | 2^{nd} Inner Shell (26.6 wt %): 96.9 MMA/3.1 ALMA |
| | Outer Shell (46.4 wt %): 77.3 MMA/20.0 ALMA/2.7 SSS |

MMA = methyl methacrylate
MAA = methacrylic acid
SSS = sodium styrene sulfonate
DVB = divinylbenzene
Sty = styrene
*Comparative

For exemplary voided latex particle P-E9, 796.9 grams (g) deionized water was added to a 3-liter, 4-neck round bottom flask equipped with overhead stirrer, thermocouple, heating mantle, adapter inlet, Claisen head fitted with a water condenser and nitrogen inlet, and heated to 84° C. under nitrogen. To the heated water was added 0.30 g acetic acid, 1.70 g sodium persulfate in 15.5 g of deionized water followed by the addition of 69.1 g of aqueous dispersion of 31% solids poly(MMA/MAA//60/40) acrylic seed (core) polymer, having an average particle diameter of approximately 185 to 205 nm. To this heated mixture at 82° C., a monomer emulsion containing 70 g deionized water, 2.1 g aqueous solution of 23% sodium dodecylbenzenesulfonate (SDBS), 91.6 g MMA, 8.9 g BMA and 3.1 g MAA was metered in over 90 minutes followed by a deionized water rinse. Next, a solution of 0.65 g sodium persulfate in 32.8 g deionized water was added over 90 minutes and the reaction temperature was raised to 90° C. concurrent with the addition of a second monomer emulsion containing 70 g deionized water, 1.9 g aqueous solution of 23% SDBS, 118.6 g MMA, 0.9 g ALMA and 0.70 g linseed oil fatty acid over 30 minutes. At the completion of addition of the second monomer emulsion, 9.6 g aqueous 28% ammonium hydroxide in 23 g of water was added, and hold for 10 min. To the reaction mixture at 91° C. was added, over 60 minutes, a third monomer emulsion containing 67.2 g deionized water, 4.7 g aqueous solution of 23% SDBS, 165.7 g ALMA, 49.7 g of MMA, and 6.1 g of sodium styrene sulfonate, followed by a deionized water rinse. The reactor contents were held at 91° C. for 30 minutes, then 5.8 g of aqueous solution containing 0.10 g of FeSO4.7H2O and 0.10 g of versene was added followed by the concurrent addition over 60 minutes of 3.3 g of t-butylhydrogen peroxide (70%) in 19.0 g of deionized water and 1.7 g isoascorbic acid in 19.0 g deionized water, to the reactor maintained at 91° C. The latex was cooled to room temperature and then filtered. All other exemplary and comparative particles were prepared substantially as described above, with the appropriate changes in monomer amounts as recited in Table 2.

All other exemplary and comparative particles were prepared substantially as described above, with the appropriate changes in monomer amounts as recited in Table 2.

### Example 2

### Characterization of Exemplary and Comparative Latex Particles

Voided latex particles as prepared in Example 1 were evaluated for particle size and percent void fraction, as shown in Table 3.

TABLE 3
Characterization of Latex Particles

| Sample | Particle Size (nm) | % Void Fraction |
|---|---|---|
| P-E1 | 443 | 17.7 |
| P-E2 | 431 | 5.2 |
| P-E3 | 404 | 14.7 |
| P-E4 | 401 | 19.0 |
| P-E5 | 418 | 22.4 |
| P-E6 | 385 | 8.5 |
| P-E7 | 328 | 17.5 |
| P-E8 | 303 | 23.0 |
| P-E9 | 575 | 31.7 |
| P-E10 | 581 | 32.9 |
| P-E11 | 872 | 33.3 |
| P-C1* | 320 | 30.7 |
| P-C2* | 302 | 25.9 |
| P-C3* | 294 | 28.3 |

*Comparative

The particle size was measured using a Brookhaven BI-90. The percent void fraction of the latex particles was measured by making a 10% by weight dispersion of each sample with propylene glycol, which was then mixed and poured into a weight-per-gallon cup which was capped and weighed. A 10% water blank was also measured, and the difference in the weight was used to calculate the density of the sample, from which the percent void fraction was determined.

### Example 3

### Preparation of Exemplary Sunscreen Formulations

Exemplary sunscreen formulations according to the present invention contain the components recited in Table 4.

TABLE 4
Exemplary Sunscreen Formulations

| | | |
|---|---|---|
| | | S-E1, S-E4, S-E5, S-E7, S-E8, |
| Trade Name | INCI | S-E9, S-E10, S-E11 (pbw) |

Phase A

| | | |
|---|---|---|
| — | DI Water | q.s. to 100 |
| ACULYN 33¹ | Acrylates copolymer | 3.33 |
| — | Glycerin | 1.00 |
| EDTA | Ethylene-diamine-tetraacetic acid tetrasodium salt | 0.10 |

Phase B

| | | |
|---|---|---|
| Escalol 557² | Octyl methoxy-cinnamate | 6.00 |
| Escalol 567² | Benzo-phenone-3 | 2.00 |
| Ceraphyl 41² | (C₁₂-C₁₅)alkyl lactate | 2.00 |
| EPITEX 66¹ | Acrylates copolymer | 1.50 |
| Dow Corning 345 Fluid³ | Cylco-methicone | 2.00 |
| — | Stearic acid | 1.50 |

Phase C

| | | |
|---|---|---|
| — | Triethanol-amine | 0.85 |

Phase D

| Polymer | S-E1 | S-E4 | S-E5 | S-E7 | S-E8 | S-E9 | S-E10 | S-E11 |
|---|---|---|---|---|---|---|---|---|
| P-E1 | 5.00 | — | — | — | — | — | — | — |
| P-E4 | — | 5.00 | — | — | — | — | — | — |
| P-E5 | — | — | 5.00 | — | — | — | — | — |
| P-E7 | — | — | — | 5.00 | — | — | — | — |
| P-E8 | — | — | — | — | 5.00 | — | — | — |
| P-E9 | — | — | — | — | — | 5.00 | — | — |
| P-E10 | — | — | — | — | — | — | 5.00 | — |
| P-E11 | — | — | — | — | — | — | — | 5.00 |
| Total | 100 | 100 | 100 | 100 | 100 | 100 | 100 | 100 |

¹Available from The Dow Chemical Company
²Available from International Specialty Products
³Available from Dow Corning

The exemplary sunscreen formulations were prepared by mixing Phase A components and heating to 75° C. In a separate vessel Phase B components were mixed together and heated to 75° C. With adequate agitation, Phase B was mixed into Phase A. After complete mixing, Phase C was added to the A/B mixture and the mixture was then cooled to 40° C., while maintaining agitation. When the mixture was 40° C. or lower, Phase D (latex particles) was added as dispersion, having been prepared by emulsion polymerization. The acrylates copolymer (as ACULYN 33) was added to the composition to provide thickening; glycerin was added as a humectant; tetrasodium EDTA (ethylenediamine tetraacetic acetate) was added for mineral ion control; octylmethoxycinnamate and benzophenone-3 (as Escalol 557 and Escalol 567, respectively) were added as UV radiation-absorbing agents; (C₁₂-C₁₅)alkyl lactate (as Ceraphyl 41) was added as an emollient and excipient; acrylates copolymer (as Epitex 66) was added as a waterproofing agent and a film-former; cyclomethicone (as Dow Corning 345 Fluid) was added as an emollient and excipient; stearic acid was added as the emulsifier; and triethanolamine was added as a neutralizing agent for both the stearic acid and the acrylates copolymer.

### Example 4

### Preparation of Comparative Sunscreen Formulations

Comparative sunscreen formulations according to the present invention contain the components recited in Table 5.

TABLE 5
Comparative Sunscreen Formulations

| | | | | |
|---|---|---|---|---|
| | | S-C1 | S-C2 | S-C3 |
| Trade Name | INCI | (pbw) | (pbw) | (pbw) |

Phase A

| | | | | |
|---|---|---|---|---|
| — | DI Water | q.s. to 100 | q.s. to 100 | q.s. to 100 |
| ACULYN 33¹ | Acrylates copolymer | 3.33 | 3.33 | 3.33 |
| — | Glycerin | 1.00 | 1.00 | 1.00 |
| EDTA | Ethylene-diamine-tetraacetic | 0.10 | 0.10 | 0.10 |
| | acid tetrasodium salt | | | |

Phase B

| | | | | |
|---|---|---|---|---|
| Escalol 557² | Octyl methoxy-cinnamate | 6.00 | 6.00 | 6.00 |
| Escalol 567² | Benzo-phenone-3 | 2.00 | 2.00 | 2.00 |
| Ceraphyl 41² | (C₁₂-C₁₅)alkyl lactate | 2.00 | 2.00 | 2.00 |
| EPITEX 66¹ | Acrylates copolymer | 1.50 | 1.50 | 1.50 |
| Dow Corning | Cylco-methicone | 2.00 | 2.00 | 2.00 |
| 345 Fluid³ | | | | |
| — | Stearic acid | 1.50 | 1.50 | 1.50 |

Phase C

| | | | | |
|---|---|---|---|---|
| — | Triethanol-amine | 0.85 | 0.85 | 0.85 |

Phase D

| | | | | |
|---|---|---|---|---|
| P-C1 | — | 5.00 (solids) | — | — |
| P-C2 | — | — | 5.00 (solids) | — |
| P-C3 | — | — | — | 5.00 (solids) |

| | | | |
|---|---|---|---|
| Total | 100 | 100 | 100 |

¹Available from The Dow Chemical Company
²Available from International Specialty Products
³Available from Dow Corning

The comparative sunscreen formulations were prepared substantially as described in Example 3.

### Example 5

### SPF Boost Heat Aging Study of Exemplary and Comparative Sunscreen Formulations

Exemplary and comparative sunscreen formulations as prepared in Examples 3 and 4 were evaluated for the capacity to retain the ability to absorb UV radiation after heat aging by measuring the sun protection factor (SPF) of the test formulations. The SPF was measured using a UV-2000S with an integrating sphere and SPF Operating Software supplied by LabSpheres (North Sutton, N.H., USA). The UV-20005 measures the UV absorbance of a sample over UV radiation Wavelengths (290-400 nm for each sample) and calculates an SPF value based on this UV absorbance spectrum. The following procedure for measuring SPF was used.

The compositions prepared were coated at a level of 7 milligram, on a 5 cm by 5 cm PMMA plate using a wire round rod. The SPF values were measured initially, after 2 weeks of storage at 45° C., and after 4 weeks of storage of the formulated samples at 45° C. The “Control” was also measured and stored in the same manner. The SPF Boost % values were calculated as follows:

$$S P F boost \% = \frac{\begin{matrix}S P F of sunscreen with voided latex particles - \\ S P F of Control\end{matrix}}{S P F of Control} \times 100 \%$$

where SPF is the measured value of the “sample” and “Control” at a given time (i.e., initial, 2 weeks, or 4 weeks) and at a given storage temperature (45° C.).

The accelerated aging tests described herein are believed to approximate the expected shelf-life for commercial formulations (containing latex particles of the present invention) stored at ambient temperatures: for example, 2 weeks at 45° C. is an estimate of shelf-life after 3 months, and 4 weeks at 45° C. is an estimate of shelf-life after 6 months.

The SPF Boost Ratio (SBR) after a certain period of heat aging of the voided latex particles is calculated as follows:

$$S B R = \frac{S B {F}_{ha}}{S B {F}_{i}}$$

where SBR is a measure of SPF boost efficacy of the voided latex particles in comparison to S-Cl. Samples having a SBR≥1.1 means that such samples outperform comparative example S-Cl; samples having a SBR≤0.9 indicates that such samples underperform comparative example S-Cl; samples having an SBR between 0.9 and 1.1 indicates that such samples perform on par with comparative example S-Cl. The decrease of the boost ratio along with the heat aging time is an indication of the poor heat stability of the voided latex particle. The results of the SBR study are shown in Table 6.

TABLE 6
SPF Boost Ratio of Exemplary and Comparative
Sunscreen Formulations

| | | SBR at 2 weeks | SBR at 4 weeks |
|---|---|---|---|
| Sample | SBR Initial | (45° C.) | (45° C.) |
| S-E1 | 0.35 | 0.67 | 0.69 |
| S-E4 | 0.49 | 0.88 | 0.79 |
| S-E5 | 0.57 | 0.55 | 0.57 |
| S-E7 | 0.40 | 0.54 | 0.58 |
| S-E8 | 0.69 | 0.57 | 0.71 |
| S-E9 | 0.95 | 1.36 | 1.16 |
| S-E10 | 0.92 | 0.94 | 1.06 |
| S-E11 | 0.63 | 0.69 | 0.44 |
| S-C1* | 1.0 | 1.0 | 1.0 |
| S-C2* | 0.71 | 0.67 | 0.64 |
| S-C3* | 0.56 | 0.35 | 0.33 |

*Comparative

The results demonstrate that exemplary sunscreen formulations prepared according to the present invention provide an SPF Boost Retention after heat aging on par with, if not better than, comparative sunscreen formulations.

### Example 6

### Opacity Study of Exemplary and Comparative Sunscreen Formulations

Exemplary and comparative sunscreen formulations as prepared in Example 3 and Example 4 were evaluated for opacity. Opacity was measured using a reflectometer (NOVO Shade DUO) with 45°/0° geometry in shade mode. The sunscreen formulations were drawn down on a Lenate form 5C opacity chart using a 3 mile BYK wet film bird type film applicator across the white and black area. The film was allowed to dry at room temperature overnight prior to the measurements. The reflectance was measured on 5 locations of the film on the white area, and the average reflectance was calculated based on these 5 values. The same type of measurement was conducted on the film on the black area of the chart, and the average reflectance was calculated. The reflectance ratio between the black area and white area was calculated by taking the ratio of the two averaged values. The ratio is a measure of the whitening effects of the hollow spheres in a sunscreen formulation. The lower the number, the less opacity that is associated with the formulation. The results of the opacity measurements are shown in Table 7.

TABLE 7
Opacity of Exemplary and Comparative Sunscreen Formulations

| Sample | Opacity |
|---|---|
| S-C1* | 0.362 |
| S-E4 | 0.161 |
| S-E9 | 0.301 |
| S-E11 | 0.307 |

*Comparative

The results of the whitening (opacity) study demonstrate that exemplary sunscreen formulations prepared according to the invention provide a lower whitening effect than comparative sunscreen formulations.

What is claimed is:

1. A personal care composition comprising:
(A) voided latex particles comprising
(i) a core polymer comprising polymerized units derived from (a) 20 to 60 weight % of monoethylenically unsaturated monomers containing at least one carboxylic acid group, based on the total weight of the core polymers, and (b) 40 to 80 weight % of non-ionic ethylenically unsaturated monomers, based on the total weight of the core polymers;
(ii) at least one inner shell polymer comprising (a) 90 to 99.5 weight % of non-ionic ethylenically unsaturated monomers, based on the total weight of the inner shell polymer(s), and (b) 0.5 to 10 weight % of aliphatic monomers selected from the group consisting of allyl acrylate, allyl methacrylate, and mixtures thereof, based on the total weight of the inner shell polymer(s); and
(iii) an outer shell polymer comprising polymerized units derived from (a) 20 to 45 weight % of non-ionic ethylenically unsaturated monomers, based on the total weight of the outer shell polymer, and (b) 55 to 90 weight % of aliphatic monomers selected from the group consisting of allyl acrylate, allyl methacrylate, and mixtures thereof, based on the total weight of the outer shell polymer; and
(B) at least one UV absorbing agent,
wherein the voided latex particles are present in an amount of from 0.5 to 20 weight %, based on the total weight of the composition, and wherein the voided latex particles contain a void and have a particle size of from 400 nm to 800 nm, and wherein the voided latex particles contain less than 10 weight % of styrene, based on the total weight of the particle.

2. The personal care composition of claim 1, wherein the voided latex particles are substantially free of styrene.

3. The composition of claim 1, wherein the aliphatic monomers are present in the outer shell polymer in an amount of from 57.5 to 80 weight %, based on the total weight of the outer shell polymer.

4. The composition of claim 1, wherein the at least one shell comprises a first inner shell polymer and a second inner shell polymer.

5. The personal care composition of claim 1, wherein the non-ionic ethylenically unsaturated monomers of the outer shell polymer comprise a monomer selected from the group consisting of acrylonitrile, (meth)acrylamide, methyl (meth)acrylate, ethyl (meth)acrylate, n-butyl (meth)acrylate, i-butyl (meth)acrylate, t-butyl (meth)acrylate, cyclo-hexyl (meth)acrylate, 2-ethylhexyl (meth)acrylate, lauryl (meth)acrylate, oleyl (meth)acrylate, palmityl (meth)acrylate, stearyl (meth)acrylate, iso-bornyl (meth)acrylate, and mixtures thereof.

6. The composition of claim 1, wherein
the monoethylenically unsaturated monomers containing at least one carboxylic acid group of the core polymer comprise a monomer selected from the group consisting of (meth)acrylic acid, (meth)acryloxypropionic acid, itaconic acid, aconitic acid, maleic acid, maleic anhydride, fumaric acid, cronotic acid, citraconic acid, maleic anhydride, monomethyl maleate, monomethyl fumarate, monomethyl itaconate, and mixtures thereof, and
the non-ionic ethylenically unsaturated monomers of the core polymer comprise a monomer selected from the group consisting of ethylene, vinyl acetate, vinyl chloride, vinylidene chloride acrylonitrile, (meth)acrylamide, methyl (meth)acrylate, ethyl (meth)acrylate, n-butyl (meth)acrylate, i-butyl (meth)acrylate, t-butyl (meth)acrylate, cyclo-hexyl (meth)acrylate, 2-ethylhexyl (meth)acrylate, lauryl (meth)acrylate, oleyl (meth)acrylate, palmityl (meth)acrylate, stearyl (meth)acrylate, and mixtures thereof.

7. The personal care composition of claim 1, wherein the outer shell polymer further comprises polymerized units derived from 0.1 to 5 weight % of a monoethylenically unsaturated monomer containing at least one carboxylic acid group.

8. The personal care composition of claim 1, wherein the outer shell polymer further comprises polymerized units derived from 0.1 to 5 weight % of a monoethylenically unsaturated monomer containing at least one “non-carboxylic” acid group.

9. A method for protecting skin from UV damage, comprising topically administering to the skin an effective amount of a personal care composition comprising:
(A) voided latex particles comprising
(i) a core polymer comprising polymerized units derived from (a) 20 to 60 weight % of monoethylenically unsaturated monomers containing at least one carboxylic acid group, based on the total weight of the core polymers, and (b) 40 to 80 weight % of non-ionic ethylenically unsaturated monomers, based on the total weight of the core polymers;
(ii) at least one inner shell polymer comprising (a) 90 to 99.5 weight % of non-ionic ethylenically unsaturated monomers, based on the total weight of the inner shell polymer(s), and (b) 0.5 to 10 weight % of aliphatic monomers selected from the group consisting of allyl acrylate, allyl methacrylate, and mixtures thereof, based on the total weight of the inner shell polymer(s); and
(iii) an outer shell polymer comprising polymerized units derived from (a) 20 to 45 weight % of non-ionic ethylenically unsaturated monomers, based on the total weight of the outer shell polymer, and (b) 55 to 90 weight % of aliphatic monomers selected from the group consisting of allyl acrylate, allyl methacrylate, and mixtures thereof, based on the total weight of the outer shell polymer; and
(B) at least one UV absorbing agent,
wherein the voided latex particles are present in an amount of from 0.5 to 20 weight %, based on the total weight of the composition, and wherein the voided latex particles contain a void and have a particle size of from 400 nm to 800 nm, and wherein the voided latex particles contain less than 10 weight % of styrene, based on the total weight of the particle.

10. A method for boosting the SPF or UV absorption of a sunscreen composition comprising adding to said composition from 0.5 to 20 weight % of voided latex particles, based on the total weight of the composition, wherein the voided latex particles comprise:
(A) voided latex particles comprising
(i) a core polymer comprising polymerized units derived from (a) 20 to 60 weight % of monoethylenically unsaturated monomers containing at least one carboxylic acid group, based on the total weight of the core polymers, and (b) 40 to 80 weight % of non-ionic ethylenically unsaturated monomers, based on the total weight of the core polymers;
(ii) at least one inner shell polymer comprising (a) 90 to 99.5 weight % of non-ionic ethylenically unsaturated monomers, based on the total weight of the inner shell polymer(s), and (b) 0.5 to 10 weight % of aliphatic monomers selected from the group consisting of allyl acrylate, allyl methacrylate, and mixtures thereof, based on the total weight of the inner shell polymer(s); and
(iii) an outer shell polymer comprising polymerized units derived from (a) 20 to 45 weight % of non-ionic ethylenically unsaturated monomers, based on the total weight of the outer shell polymer, and (b) 55 to 90 weight % of aliphatic monomers selected from the group consisting of allyl acrylate, allyl methacrylate, and mixtures thereof, based on the total weight of the outer shell polymer; and
(B) at least one UV absorbing agent,
wherein the voided latex particles are present in an amount of from 0.5 to 20 weight %, based on the total weight of the composition, and wherein the voided latex particles contain a void and have a particle size of from 400 nm to 800 nm, and wherein the voided latex particles contain less than 10 weight % of styrene, based on the total weight of the particle.
